(19) United States
(12) Patent Application Publication
(10) Pub. No.: US 2008/0009749 A1
(43) Pub. Date: Jan. 10, 2008

(54) Curved needle assembly for subcutaneous light delivery

(76) Inventors: Theodore Philip Delianides, Boulder, CO (US); Adriaan Gerard Verheij, Evergreen, CO (US)

Correspondence Address: Theodore Philip Delianides, Suite K, 3131 Indian Rd., Boulder, CO, 80301, US

(21) Appl. No.: 11/473,095

(22) Filed: Jun. 22, 2006

(51) Int. Cl. A61B 6/00

(52) U.S. Cl. 600/476

(57) ABSTRACT

In this invention, a substantially straight needle containing one or more optical fibers is bent into a curved shape, typically including a helical portion, after the insertion of optical fibers into the needle. This curved needle, with its integral optical fibers, allows the subcutaneous delivery of light to a tissue-under-test. The preferred embodiment of this apparatus performs three functions: subcutaneous delivery of light to a tissue-under-test, attachment to the tissue-under-test, and subcutaneous electrical contact. In this invention the optical fibers are inserted into the needle prior to needle bending which prevents fiber breakage because the needle is not curved or in a helical form and therefore provides no resistance to the insertion of the fibers.

### STATEMENT REGARDING FEDERALLY SPONSORED RESEARCH OR DEVELOPMENT

This invention was made with government support under R44 HL081866 awarded by the National Institutes of Health. The government has certain rights in the invention.

### BACKGROUND OF THE INVENTION

The present invention relates generally to the field of fetal monitoring and more specifically to the photoplethysmographic measurement of oxygen saturation and heart rate from a fetus during labor and delivery.

Pulse oximeters are commonly used in adult and neonatal care to provide a measurement of arterial oxygen saturation. A pulse oximetry system typically consists of a sensor which is applied to the patient, a monitor on which the measurements of arterial oxygen saturation are displayed, and a cable which connects the sensor to the monitor. The sensor typically contains light emitting diodes whose output light is incident on the surface of the tissue-under-test and a photodiode detector that measures the intensity of the light exiting the tissue-under-test at the sensor site.

The use of pulse oximetry has recently been expanded to include its use on a fetus during labor and delivery. U.S. Pat. No. 5,228,440 reveals a fetal pulse oximetry sensor which is intended to be positioned on the fetal cheek or side of the fetal head. This sensor does not adhere to the fetus and is therefore sensitive to changing position with respect to the fetus as a result of contractions during labor and progression of the fetus through the birth canal. This movement of the sensor with respect to the tissue-under-test often results in a loss of signal necessitating repositioning of the sensor. In addition, the application of the emitter and the detector to the same surface of the tissue-under-test, versus placement across the tissue-under-test as when the emitter and the detector in the sensor are placed on opposite sides of a finger, allows the possibility of light being shunted directly from the emitter to the detector without passing through the tissue under test. This can cause the fetal pulse oximetry readings to be erroneous.

Alternate methodologies for fetal pulse oximetry have been considered that make use of a modified version of the fetal spiral electrode, a device designed and manufactured for the measurement of the fetal electrocardiogram (ECG). This spiral ECG electrode is disclosed in FIGS. 8, 9, and 10 of U.S. Pat. No. 3,827,428. The spiral, or more accurately “helical”, ECG electrode in combination with fetal pulse oximetry has been presented in a number of different potential configurations.

In U.S. Pat. No. 5,154,175 the helical electrode is used to hold the emitter and detector elements flush against the fetal scalp, which is the tissue-under-test. While this sensor remains fixed with relation to the fetus, it still has the problem that both the emitter and the detector lay on the same surface of the tissue-under-test. This allows the possibility of errors in readings caused by light being shunted directly from the emitter to the detector without passing into or through the tissue-under-test.

Two patent documents disclose a potential solution to this problem, U.S. Pat. No. 5,361,757 and U.S. Patent Application Publication No. 2005/0283059 A1. In the first of these two publications, the emitters are light emitting diodes (LEDs) that are positioned at a window in the helical needle and therefore, when the sensor is in position in the fetal scalp, the light is emitted into the tissue-under-test and, after emerging from the tissue, is incident on a detector located at the base of the sensor positioned on the surface of the fetal scalp. Publication No. 2005/0283059 A1 reveals a slightly different arrangement in which both the LEDs and the detector are positioned in the helical needle and the light is transmitted from the emitters directly across to the detector subcutaneously, because both elements are located under the surface of the skin once the sensor is in place on the fetus.

The problems common to both of these LED based solutions include: the difficulty of creating and handling miniscule circuit boards and optical elements; threading these active elements through a hollow needle; aligning the active elements with the windows in the needle; creating windows in the side of a hollow needle; and potting the active elements into place in the needle window or windows while maintaining their proper alignment within the window. The methodology is expensive to build, difficult to manufacture, and creates a very short pathlength for the light to pass through the tissue-under-test, which results in poor signal-to-noise ratios.

One potential solution to these problems is to thread an optical fiber down the hollow needle. This optical fiber would provide a means for propagation of light down the length of the needle and for final emission at the end of the needle. This would eliminate the need to cut microscopic windows in the side of a hollow stainless steel needle or to create miniature circuit boards that would have to be threaded through the needle.

The idea of using optical fibers in a needle has been considered by more than one inventor. It is mentioned in U.S. Patent Application Publication No. 2005/0283059 A1 as an alternate methodology to the use of LEDs in the needle. The use of optical fibers in a helically shaped needle was also revealed in the earlier 1987 U.S. Pat. No. 4,658,825 where the use of optical fibers in a helical needle assembly was disclosed for the measurement of pH.

None of the prior art, however, discusses how to successfully create a small diameter helical needle containing optical fibers. In fact there appears to have been no fiber-based helical needle products successfully manufactured or sold in the field of medical monitoring. This may be due, in no small part, to the difficulty of creating such an apparatus without shattering the fibers internal to the needle. Forcing one or more optical fibers down a helical needle inevitably fractures the fibers due to the stresses of the insertion process. The leading edge of the cut fiber also catches on the inside of the needle, thus resisting the insertion process. In addition, in helical needles of the configuration and size typically used for fetal ECG monitoring, the bend radius that the fiber must undergo can be close to the minimum-sustainable bend radius for a glass fiber, thus considerably adding to the likelihood of fiber breakage.

Another aspect of this problem is how to shape the distal end of the needle. To facilitate insertion into living tissue, the distal end of the needle is typically cut or polished to a beveled, sharp point, similar to the tip of a hypodermic needle When a needle is bent into a small-diameter helical shape the internal fibers are under a great deal of bending stress. As the distal end of the needle is cut or polished to form the required beveled point, the scoring associated with polishing or cutting will cause cracking in the stressed fibers.

The solution to the problem of how to create a helical needle containing one or more undamaged optical fibers capable of carrying light to or from the tissue-under-test is the subject of this invention.

### BRIEF SUMMARY OF THE INVENTION

The object of this invention is to create a needle assembly consisting of a hollow metal tube containing one or more optical fibers. The assembly is, at least in part, in a helical configuration with the distal end shaped to a point. This pointed distal end of the needle is designed for easy insertion into living tissue.

The needle assembly is typically designed to provide three specific functions. First, it provides positive attachment to the tissue-under-test, typically the fetal scalp. The helical portion of the needle is commonly rotated into the fetal scalp, usually one full turn. This holds the sensor body firmly against the fetal scalp.

Second, the integral fibers function to deliver light below the surface of the skin to the tissue at the tip of the needle. This is a particularly advantageous configuration for photoplethysmographic measurement techniques such as pulse oximetry. In a photoplethysmographic sensor of this configuration, the emitted light must travel from the emitter, in this case the distal end of the needle, through the intervening tissue before it can be incident on the detector situated in the base of the sensor body. This eliminates the possibility of light shunting directly from the emitter to the detector as sometimes occurs in “reflectance mode” pulse oximetry sensors. With reflectance sensors, whether for fetal or adult use, light can travel directly from the emitters to the detector, without ever entering the tissue-under-test, thereby causing erroneous measurements.

The actual light source used in conjunction with the needle assembly of this invention can be located in any of a number of different places. Because the light delivery is by fiber optics, the source emitter or emitters can be located at a distance from the tip of the needle. They could be internal to the monitor, in the sensor body, or anywhere in between. For example, the emitters could be located in a connector or in a housing positioned on the sensor cable. It should also be noted that the optical fibers could provide the function of receiving the light that had passed through the tissue-under-test rather than being used to deliver the light to the tissue. The light would then be conducted via the optical fiber or fibers to one or more detectors positioned in the monitor, at the sensor connector, on the sensor cable, or in the sensor body. Such a configuration would have some disadvantages in photoplethysmography in that the detecting fiber would only pick up a small amount of the light compared to the amount that would be received by a typical photodetector conventionally used in pulse oximetry.

The third specific function of the needle assembly of this invention is to provide an electrode for ECG measurement. This assembly, installed in the appropriate sensor body, can therefore replace the conventional fetal spiral ECG electrode while providing the added function of acting as the emitter portion of the sensor for a photoplethysmographic monitor such as a pulse oximeter.

Two main elements of this invention make this a manufacturable assembly. The optical fibers are inserted into the hollow metal tube, or needle, prior to bending the tube into its curved shape. This eliminates any source of resistance while inserting the fibers into the needle. Once the fibers are in place inside the needle, the assembly is bent around a mandrel into its final form. In the current embodiment of this needle assembly there are two separate bends required. The first sweeps the needle from the straight portion that will extend through the sensor body towards the helix, and the second bend is the helical portion of the needle assembly, the portion that is rotated into the tissue-under-test.

The second element of this invention involves creating the pointed or beveled end of the needle assembly. The distal end of the needle, the end which is first inserted into the tissue-under-test, must be pointed to allow easy insertion into the tissue. To create this pointed end of the needle, the tip of the distal end is polished to create a bevel. If the polishing operation is performed anywhere on the helical portion of the assembly, however, the fibers will fracture or shatter along the beveled portion. This diminishes the optical transmissivity and could allow small pieces of the fibers to be left behind when the sensor is removed from the tissue-under-test. This fracturing occurs because the bent fibers are under stress and the polishing or cutting process used to create the bevel causes scoring of the fibers. The fibers then fracture along the score lines to relieve the bending stress in the glass.

To prevent this stress fracturing, a very small segment of the distal end of the helical portion of the needle assembly is not bent. For the size scale of a typical fetal scalp sensor helical needle, a straight segment with a length of one or two millimeters is sufficient. It is this straight portion of the assembly that is beveled; and, because the fiber or fibers inside this portion are also not bent, there is no stress in the glass and the fibers do not fracture.

While much of this discussion has been focused on glass based optical fibers, many of the same problems exist with other types of light pipes such as polymer-based optical fibers, also called plastic optical fibers. Inserting plastic fibers down a needle already bent into its final configuration would be extremely difficult given the high and ever-increasing level of friction that the fibers would create against the needle walls as they attempted to traverse the various bends.

### BRIEF DESCRIPTION OF THE SEVERAL VIEWS OF THE DRAWING

FIG. 1. Fetal Oximetry Sensor: Drawing of the fully assembled sensor head with all electrical and optical connectors.

FIG. 2A. Fibers Inserted into Unbent Needle: View showing fibers inserted into a straight needle.

FIG. 2B. Fibers in Needle after Bending: View showing fibers and needle in the curved shape.

FIG. 3. Needle Assembly, Distal End Detail: Drawing of the finished needle assembly showing detail of the polished end face.

FIG. 4. Unbent Segment at Distal End: View of the helical portion of the needle showing the unbent segment at the distal end of the needle.

### DETAILED DESCRIPTION OF THE INVENTION

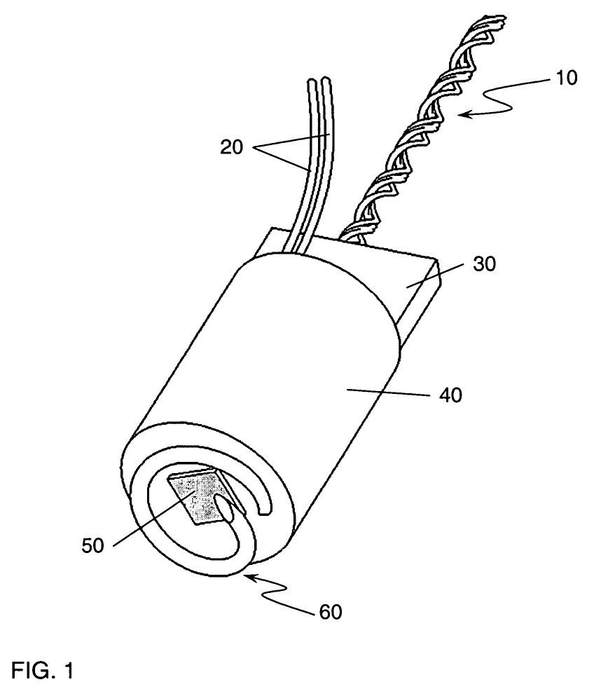

Helical needles have been in use for many years for the purpose of monitoring the fetal electrocardiogram (ECG). These devices were referred to as spiral needle electrodes or fetal spiral electrodes. This spiral electrode is a solid needle that is rotated into the fetal scalp. The spiral needle provided one of the electrodes necessary for monitoring the fetal ECG. The “spiral” needle electrode is actually helical in shape. This helically shaped electrode is similar to the one shown as item 60 on the sensor pictured in FIG. 1. In the conventional spiral needle electrode the needle was solid, not hollow, and the distal end was beveled to create a point.

A complete sensor incorporating the needle assembly of this invention is depicted in FIG. 1. This sensor would allow the monitoring of the fetal ECG in conjunction with the monitoring of fetal arterial oxygen saturation. Such a sensor would consist of both optical and electrical components. The needle assembly 60, of this invention, is a hollow metal tube, also referred to as a needle, containing one or more optical fibers. In this sensor design optical fibers 20 extend from the proximal end of the needle out through the sensor body 40 for connection to the light source or sources. The optical fibers typically serve to deliver light subcutaneously to the tissue-under-test.

The electrical conductors 10 connect to three different elements in the depicted sensor design. One conductor connects to the ECG tab electrode 30, a second conductor connects to the needle assembly 60, and two more conductors connect to the photodetector 50. The photodetector 50 is positioned against the fetal scalp, when the sensor is in use, where it can receive the light emitted subcutaneously by the optical fibers after the light passes through the tissue-under-test which in this case is the fetal scalp.

For the needle assembly 60 to be useful as an ECG electrode it must be electrically conductive and it must be electrically connected to one of the electrical conductors 10. This typically mandates that the needle be made out of a conductive metal. This also facilitates the ability to sharpen the distal end of the needle to ease insertion into the tissue-under-test.

Optical fibers have been created out of a variety of different materials including glass and plastic. Glass based optical fibers have better transmission properties than do plastic core fibers over the red and near infrared region typically used in photoplethysmographic devices such as pulse oximeters. Thus glass core optical fibers are more likely to be used in photoplethysmographic sensors. Although glass core optical fibers typically are encased in a buffer material such as a plastic, when glass fibers are inserted down a tube that is curved, the sharp leading edge of the fibers catches on the side of the tube, drastically increasing the resistance to insertion. Even in the case of plastic core fibers the friction between the plastic and the sides of the tube increases as the fiber tries to traverse further and further down a bent tube.

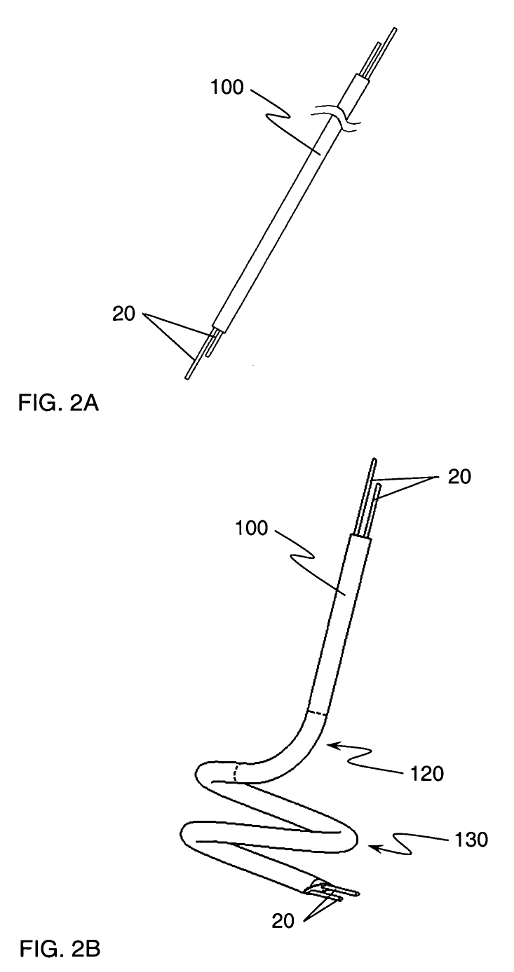

In attempting to insert one or more optical fiber down a curved needle of the shape shown in the bent needle drawing of FIG. 2B the friction between the fibers and the wall of the needle increases dramatically as the fibers progress down the needle. Eventually the friction is so great that the force necessary to advance the fibers would cause glass core optical fibers to fracture or, in the case of the plastic core optical fibers, the friction becomes so great as to cause the fiber to bend or buckle at the insertion point rather than to progress further into the needle.

To minimize the insertion force placed on the fiber or fibers as they are inserted into the needle 100, and to allow for complex bends in the needle assembly, the optical fibers 20 are inserted into the needle when the needle or hollow metal tube 100 is in a relatively unbent or substantially straight condition. In the unbent condition there is minimal friction between the fibers and the inside wall of the needle, thus allowing the fibers to be easily inserted. Fibers inserted into an unbent needle are shown in FIG. 2A. Note that it is likely that hollow metal tubes as received from the manufacturer will contain some slight bend but for the sake of this invention this is considered to be the unbent configuration if the bend is minimal and will impose little or no resistance to insertion of the fibers.

Once the optical fibers are in place in the needle, the needle and fibers can be bent into their final configuration. In the needle assembly of the preferred embodiment of this invention there are two different bends that make up the final configuration or curved shape. The first bend 120 shown in FIG. 2B is a sweep that takes the straight needle into a curve that provides a starting point for the helix. The second bend is a helix 130 which can be any number of turns or a fraction of a single turn as required.

The needle will typically be bent in a bending machine around a form or mandrel in an automated or semi-automated manner. The mandrel will ensure that each needle assembly manufactured will have close to the identical curved shape and will be designed such that no bend in the needle is tighter than the minimum bend radius of the fiber. The minimum bend radius is the shortest radius of curvature which the fiber can maintain without fracturing prior to, or during, use in the finished sensor. The bending machine will also provide a means to hold and protect the fibers from damage during bending.

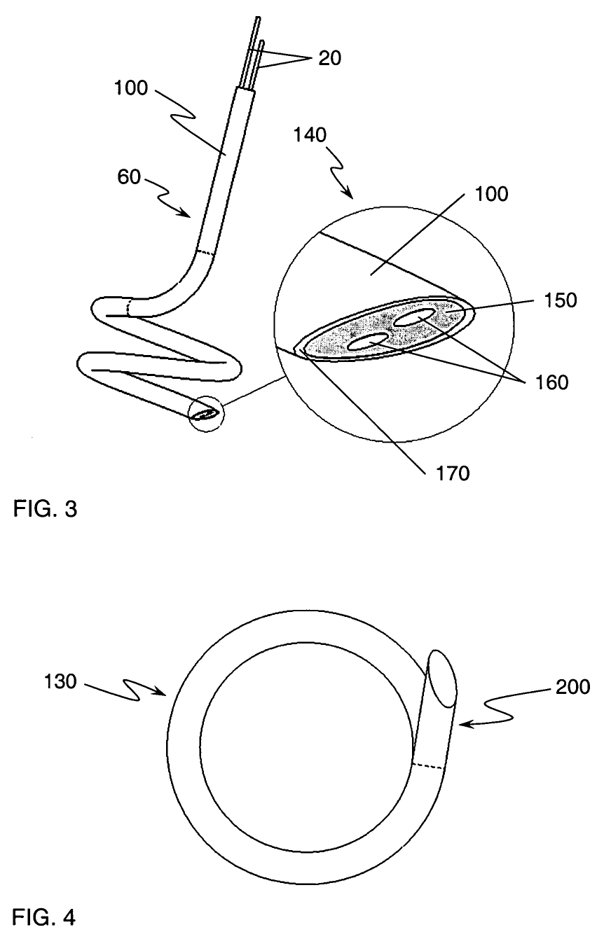

Once the needle is bent into its final curved shape the space internal to the needle, not filled by the optical fibers, is typically infused with a filler material 150 as shown in FIG. 3. Finally, the distal end of the needle is shaped for easy insertion into the tissue-under-test.

The filler material provides a number of functions including holding the fibers firmly in place within the needle, providing a sealed end surface of the needle, and adding a measure of rigidity and strength to the assembly. The filler material can be an epoxy, or other adhesive, or it may be a plastic polymer. Epoxies can be inserted into the bent needle assembly and then cured after insertion into the hardened state. A polymer may be forced into the unfilled space internal to the hollow needle by either insertion prior to polymerization of the plastic or, in the case of some thermoplastics, by warming the plastic to make it flow and then allowing it to cool and harden once it is in place. It is also possible to infuse the filler material into the needle when it is in the unbent condition and then form the needle, fibers, and filler assembly into its curved shape when the filler material is still in a malleable state.

To shape the distal end of the needle for easy insertion into tissue it is typically sharpened to a point. This operation will usually involve a cutting or polishing process or both. With the needle bent in a curve, the fibers internal to the needle can be under a great deal of stress. If the optical fibers are glass core, they will usually fracture as they are being cut or polished at an angle other than 90 degrees with respect to the long axis of the fiber.

To allow the distal end of the needle to be cut or polished at an angle without causing the fibers to fracture, a short segment of the distal end of the needle is left in the unbent state. The fiber or fibers in this portion of the needle are also in the unbent state and therefore not under stress. This unbent segment 200 of the needle assembly is shown in FIG. 4. With the filler material holding the fibers firmly in place within the needle, the distal end of the small unbent segment can be polished or cut to a point without fracturing the fiber or fibers internal to the needle.

In the preferred embodiment of this invention the cutting and polishing operation applies a bevel 140 to the distal end of the needle which brings this end to a point as shown in the enlargement of the polished needle end in FIG. 3. The cutting or polishing process creates a flat end face exposing the polished ends 160 of the fibers 20, the filler material 150, and the needle wall 170. Only a very small segment of the distal end of the needle has to be left in the unbent configuration to avoid stressing the fibers and prevent fractures during cutting or polishing of the tip. This portion of the needle 200 only deviates from the helical form 130 very slightly as shown in FIG. 4, thus it does not interfere with smooth insertion into the tissue-under-test. This polishing or cutting brings the needle and fiber ends to a beveled finish, and, with the filler material at the end face of the needle also polished to the same angle, the end face is a flat surface impervious to interstitial fluid or blood.

The previous discussion of the invention has been presented for the purposes of illustration and description. The description is not intended to limit the invention to the form disclosed herein. Variations and modifications commensurate with the above are considered to be within the scope of the present invention. The embodiment described herein is further intended to explain the best mode presently known of practicing the invention and to enable others skilled in the art to utilize the invention as such, or in other embodiments, and with the particular modifications required by their particular application or uses of the invention. It is intended that the appended claims be construed to include alternative embodiments to the extent permitted by the prior art.

1. An apparatus for subcutaneous delivery of light to a tissue-under-test comprising:
a needle containing one or more optical fibers,
where the needle takes on a curved shape,
and where the optical fibers are inserted into the needle prior to the needle being bent into the curved shape.

2. The apparatus of claim 1 wherein the curved shape of the needle includes a segment of the needle in the form of a helix.

3. The apparatus of claim 1 wherein the needle provides the additional functionality of creating a subcutaneous electrical contact

4. The apparatus of claim 1 wherein the bend radius of any bend in the needle is constrained to be larger than the minimum allowable bend radius of the one or more optical fibers

5. The apparatus of claim 1 wherein the space internal to the needle and not occupied by the optical fibers is filled with a filler material.

6. The apparatus of claim 5 wherein the filler material is added after the needle is bent into the curved shape.

7. The apparatus of claim 5 wherein the filler material is added before the needle is bent into the curved shape and wherein the filler material is in a malleable state during bending of the needle.

8. The apparatus of claim 1 wherein a distal end of the needle is shaped for insertion into a tissue-under-test.

9. The apparatus of claim 1 wherein a small distal end segment of the needle is unbent.

10. The apparatus of claim 9 wherein a distal end of the unbent segment of the needle is shaped for insertion into a tissue-under-test.

11. An apparatus for subcutaneous delivery of light to a tissue-under-test comprising:
a needle containing one or more optical fibers,
where the needle takes on a curved shape some portion of which is in the shape of a helix,
where the optical fibers are inserted into the needle prior to the needle being bent into the curved shape,
where the space inside the needle not taken up by the one or more optical fibers is filled with a filler material,
and wherein a distal end of the needle is shaped for insertion into a tissue-under-test.

12. A method for manufacturing a curved needle containing one of more optical fibers for delivery of light to a tissue-under-test comprising the steps of:
inserting the one or more optical fibers into a needle,
bending the needle into a curved shape after the one or more optical fibers have been inserted into the needle.

13. The method of claim 12 wherein the curved shape of the needle includes a segment of the needle in the form of a helix.

14. The method of claim 12 wherein the needle provides the additional functionality of creating a subcutaneous electrical contact

15. The method of claim 12 wherein the bend radius of any bend in the needle is constrained to be larger than the minimum allowable bend radius of the one or more optical fibers.

16. The method of claim 12 including the step of adding a filler material to fill the space between needle and the one or more optical fibers.

17. The method of claim 16 wherein the filler material is added after the needle is bent into the curved shape.

18. The method of claim 16 wherein the filler material is added before the needle is bent into the curved shape but wherein the filler material remains in a malleable state during bending of the needle.

19. The method of claim 12 including the step of shaping a distal end of the needle for insertion into the tissue-under-test.

20. The method of claim 12 including the step of leaving a small distal end segment of the needle in the unbent configuration.

21. The method of claim 20 including the step of shaping a distal end of the unbent segment of the needle for insertion into a tissue-under-test.

22. A method for manufacturing a curved needle containing one or more optical fibers for delivery of light to a tissue-under-test comprising the steps of:
inserting the one or more optical fibers into a needle,
bending the needle into a curved shape after the one or more optical fibers have been inserted into the needle,
adding a filler material to the fill the space inside the needle not taken up by the one or more optical fibers,
shaping a distal end of the needle for insertion into a tissue-under-test.
